(19) United States
(12) Patent Application Publication
(10) Pub. No.: US 2010/0278949 A1
(43) Pub. Date: Nov. 4, 2010

(54) PRE-PROCEDURE MEAL REGIMEN

(76) Inventor: JEFFERY DALE SCOTT, Stillwater, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 12/772,625

(22) Filed: May 3, 2010

Related U.S. Application Data
(60) Provisional application No. 61/174,542, filed on May 1, 2009.

(51) Int. Cl. A61K 36/48; A61K 31/08; A61P 1/10

(52) U.S. Cl. 424/757; 514/723

(57) ABSTRACT

A colon prep method and kit for facilitating a method for evacuating a colon prior to a medical procedure, wherein the method minimizes potential patient discomfort. Three selected meals and a plurality of selected snacks are ingested on a day prior to the medical procedure. The meals include solid food items taken with or incorporating a stimulant laxative, such as senna, incorporated therein. A liquid having a PEG laxative, such as PEG-3350, is provided with or incorporated therein, preferably via a powdered drink mix. The stimulant laxative and the PEG laxative work in synergy. A further optional snack may be provided on the day of the medical procedure, preferably ingested approximately four hours before the medical procedure.

### CROSS REFERENCE TO RELATED APPLICATIONS

This application claims the priority of U.S. Provisional Patent Application No. 61/174,452 entitled “PRE-PROCEDURE MEAL REGIMEN,” filed May 1, 2009, the contents of which are hereby incorporated by reference.

### FIELD OF THE INVENTION

The invention relates to a method and kit for facilitating the method of preparing a colon for a medical procedure. More particularly, the invention utilizes a pre-packaged kit having selected food and drink provided with, or laced, with laxatives that preclude a patient having to fast and which facilitates proper colon preparation.

### BACKGROUND OF THE INVENTION

The American Cancer Society estimates about 110,000 new cases of colorectal cancer will be diagnosed in 2008. Overall, the lifetime risk is 1 in 19. Colorectal cancer is the second leading cause of cancer death in the U.S., causing approximately 50,000 deaths estimated for 2008. The death rate, however, has been dropping for more than 20 years due to polyp detection. Colonoscopy is the gold standard in diagnosing and removing precancerous polyps, thereby preventing colon cancer. Rates for screening have perennially been poor, partially related to availability of proceduralists, referral patterns of physicians, but also largely due to patient reluctance.

Colonoscopies have traditionally had a poor name due to cost, discomfort and most importantly, the prep. The cost has steadily declined with coverage by Medicare and insurance companies. Discomfort has also been addressed with IV anesthetics and the use of Propofol. Colonoscopy preps, however, have not made significant advances in over 40 years.

Adequate colon exams require adequate visualization of the colonic mucosa. Bowel preparations are evaluated on three criteria: efficacy in cleansing, safety, and tolerability. Unfortunately no preparation today is the clear winner in all three categories.

Laxative Types

There are three types of bowel purgatives for colonoscopy: stimulants, osmotic agents, and polyethylene glycol based solutions (PEG). Stimulants include senna, cascara, bisacodyl, and sodium picosulfate. Osmotic preps include sodium phosphate (NaP, a hypertonic salt solution), magnesium citrate and mannitol. Osmotic agents and non-absorbable large volume lavages (PEG) taken with “clear liquid diets” have been the standard for colon preparations for years. They are, however, associated with a multitude of complaints including bloating, cramping, nausea, vomiting, bad taste, hunger, weakness, and the inability to understand prep directions. These complaints represent an important barrier to compliance with colonoscopy. Suboptimal bowel preparation leads to prolonged procedure times, lower rates of cecal intubation, reduced screening intervals, higher screening costs and possibly increased risk for procedural complications. Inadequate cleansing of the colon, reported to occur in approximately 27% of all exams, results in missed adenomas as reported by Froehlich F., in Gastrointestinal Endoscopy 2005, Volume 61, Pages 378-384. Also 17-30% of suboptimal colonoscopies in different trials were because of inadequate bowel preparation.

1. Fiber. Much of the prior art, with respect to bowel movements deals with ingestible fiber or bulking agents in the form of psyllium, methylcellulose, polycarbophil, calcium polycarbophil, bran, malt, pectin, soup extract, karaya guar gum and various mixtures. These components cause a change in the composition and fluid content of stool but do not cause a cathartic effect with total evacuation of colon contents. In fact, one of their benefits is minimal to no diarrhea. They are also much slower in onset of bowel changes. Stool softeners offer an intermediate between bulk and stimulant laxatives. Dioctyl sulfosuccinate is the active ingredient in some. It is an anionic medicinal surfactant possibly acting as an intestinal secretogogue and enhancing retention of luminal fluid increasing fecal fluid content. This, again, is typically used in the occasional treatment of constipation but not as a diarrheagenic producing agent.

2. Senna. Stimulants include cascara and senna. Senna is a plant-derived compound from the leaves, or pods, of various species of the cassia plant. Commercial sources include the species cassia angustifolia (Tinnevella senna) and cassia acutifolia (cassia senna or Alexandria senna). Commercial concentrates range from 20-95% calcium sennosides. The senna plants are small shrubs cultivated either in Somalia, the Arabian Peninsula or near the Nile River. Tinnevella senna is obtained from cultivated plants mainly in south India and Pakistan. The active ingredient of senna was first isolated and characterized by Stoll in 1941. There are two glycosides identified and attributed to the anthraquinone family senusoids A and B. They both hydrolyze to give the aglycones sennidin A and B and two molecules of glucose. Later work also demonstrated the presence of senusoids C and D. Studies have shown that the anthraquinone glycosides pass unchanged into the colon where bacteria hydrolyzed the glycoside bond, yielding 3 anthraquinones which has a direct stimulant effect on the myenteric plexus of the bowel, resulting in smooth muscle contraction and, thus, defecation. It also helps to temporarily prevent fluid from being absorbed by the large intestine, thus contributing to softer stools. Senna has been used by people in northern Africa and southwestern Asia as a natural laxative for centuries. It was labeled a “cleansing” herb for its laxative effects. The leaves were also sometimes made into a paste and used for various skin conditions. Senna is considered a sage and effective laxative used throughout the world. It is currently sold commercially as an active ingredient in laxative products or as a sale OTC laxative to relieve occasional constipation. Senna has not however been typically used as an agent to prepare patients for endoscopic, radiologic or surgical procedures.

K. Butt and colleagues conducted a randomized blinded study to compare the efficacy and patient tolerance of high dose oral senna suspension versus a conventional sodium phosphate prep in adults undergoing elective colonoscopy. Patients either received two doses of liquid senna (158 mg each, 8 hours apart) the day before colonoscopy (N=50) or a conventional sodium phosphate regimen (48 grams; N=50). Tolerance and compliance was significantly better with the senna preparation than with the sodium phosphate with more patients reporting good tolerance (46 versus 28 patients) and fewer reporting poor tolerance (0 versus 12 patients) to the senna versus the sodium phosphate regimen. Also there was a trend toward improved preparation quality.

Another study by F. Radaelli and colleagues was published in the American Journal of Gastroenterology in December 2005 comparing oral high dose senna to standard 4 liter PEG solution. 191 patients were randomized to the senna group (24 tablets of 12 mg senna divided into 2 doses at 1:00 P.M. and 9:00 P.M.) versus 192 patients who received the standard 4 liter PEG preparation. Regardless of which prep was used, all patients were instructed to eat a low fiber diet and increase water intake on the 4th through 2nd pre-procedural days. On the day before the procedure, they were advised to eat a normal breakfast in the morning and clear liquids thereafter until 2 hours before the colonoscopy. The quality of colon cleansing, overall tolerance of the preparation and compliance were significantly better with senna. Radaelli prepped patients with 288 mg total senna in two divided doses with good colon cleansing but increased abdominal pain compared with 4 liters PEG.

3. Sodium Phosphate. Hypertonic salt solutions have been used for many years and, in fact, the first Fleet laxative was developed in 1893 by Dr. C. B. Fleet. Fleet's phosphosoda is a hypertonic saline cathartic agent that pulls water into the bowel lumen to flush the food residue from the GI tract. This influx of fluid into the bowel causes bowel distention and contractions of smooth muscle in the bowel wall. The patient may experience bloating and cramping with resultant diarrhea. It also can cause nausea and vomiting because of the salty taste and bowel contractions. More importantly, the preparation frequently causes electrolyte shifts and potentially dangerous electrolyte imbalances. In addition to electrolyte shifts, cases of an acute phosphate nephropathy have been reported causing permanent kidney damage. Calcium phosphate may form in the kidney, damaging nephrons and the structure of the kidney, possibly leading to renal failure. This has prompted a warning by the FDA in May 2006.

4. Polyethylene Glycol. Whole bowel irrigation was originally developed to cleanse the large bowel before surgery or colonoscopy. The initial electrolyte solution was partially absorbed, sometimes leading to complications. Then, a specialized isoosmolar solution called polyethylene glycol (PEG) was developed to combat this problem. PEG is a polyether with a molecular mass below 20,000 grams per mol. Polyethylene glycol has a low toxicity and is used in a variety of products. It is available in liquid and powder form and is the basis of a number of laxatives, skin creams and lubricants among other commercial products. PEG with added electrolytes is sold under the brand names Go-Lightly, MiraLax®, GlycoLax®, Tri-Lite, Co-Lyte and Half Lightly®. MiraLax® has a molecular weight of 3,350. PEG solutions have been used on millions of patients preparing for colonoscopic procedures. These preparations involve ingestion of 2-4 liters of solution with resultant diarrhea. Unfortunately ingestion of PEG is also generally accompanied by a sense of fullness, cramping, nausea, and vomiting. Patients frequently have difficulty completing the prep, thereby causing inadequate bowel cleansing.

Recently, the use of a powdered PEG-3350, or MiraLax® has been reported as a sole bowel preparation agent. MiraLax® was initially introduced as a prescription agent for the treatment of occasional constipation. After 10 years on the market, it was produced as a generic called GlycoLax® until an FDA arrangement for a 3-year marketing exclusivity agreement as OTC MiraLax® which expires in approximately the winter of 2009.

In a study by M. Arora, published in Gastroenterology and Hepatology, July 2008, 245 patients were studied who received 204 grams of powdered PEG-3350 dissolved in 32 ounces of water and taken in 3 divided doses, one hour apart with 8 ounces of water in between each dose. The colon preparations were then compared to patients receiving a standard sodium phosphate prep. A scale of 0-4 was used for colon preparation with the zero being a failed procedure due to a poor prep and 4 being completely clean. The MiraLax® group was calculated to have a mean score of 3.43 and therefore, quite favorable as a bowel preparation agent.

Prep Barriers

One-third (34%) of patients reported mid-range to severe discomfort from the preparation regimen to clear the bowel in an A.A.A.H.C. report surveying 2,000 people at 107 ambulatory surgery centers from August to November of 2007. The prep was their number one complaint. The combination of numerous gastrointestinal upsets, including taste, nausea, vomiting, bloating, and cramping associated with these agents leads to an almost universal avoidance by patients of a potentially lifesaving procedure. Patients frequently have difficulty completing the preparation but are unable to quantify their colon cleanliness. They miss work, with its associated costs, and undergo attempted colonoscopy which may be incomplete with regard to inspection of the full colon due to a poor colon cleanse. Studies have shown significant expense related to poor prep due to repeat exams or shorter surveillance intervals because of decreased confidence by the endoscopist in their colonoscopy findings.

Annual demand for colonoscopy ranges from 8-14 million and is increasing. Studies demonstrate a severe undersupply of professionals able to perform endoscopies, including gastroenterologists, surgeons, primary care physicians and physician extenders. This has been associated with a higher volume of screening colonoscopies per physician, and in some cases, a decrease in the pre-procedure instruction time spent with patients.

In a study published in the American Journal of Gastroenterology in June 2001, researchers studied pre-endoscopy education. The study included 142 patients. Cancellation of the procedure occurred in 26.3% of patients in a written instruction group versus 4.4% in a more rigorous patient education group.

In addition to inadequate pre-procedure teaching, low literacy is another cause of poor bowel preparations. In a study of 195 patients at an inner city hospital presented at “Digestive Disease Week” in 2006, 30% had poor bowel preparation, requiring a repeat exam. Another 22% had only “fair” bowel preparation, meaning small or flat lesions could be missed. A 7-minute literacy test determined 40% had low literacy and 20% marginal literacy. Those with the low literacy had a 63% rate of poor bowel preps compared with 12% of those patients with marginal or adequate literacy. The researchers concluded that better methods of explaining colonoscopy preparation must be developed.

Prep Diets

Traditionally, patients have also been placed on long periods of clear liquids while they are enduring a colon preparatory agent to ensure adequate bowel cleanliness. This “diet” first requires they understand the meaning of a clear liquid diet. While that may be obvious to some, many patients have great difficulty discerning different food products as “clear liquids”. They must then purchase and prepare clear liquids that are not a typical part of their natural dietary regimens. Lastly, the patient must have the willpower to fast despite increased hunger associated with ingestion of only clear liquids. S. McCray, R. D. and D. Balaban, M. D. summarized a series of studies in Practical Gastroenterology in November 2007 that looked into these diet regimens that may be more tolerable to patients but still produce adequate colon preparation when combined with current bowel laxatives.

Scott, et al, randomized 200 patients undergoing colonoscopy to receive a sodium phosphate oral preparation with either: 1) standard light breakfast followed by clear liquids on the day before the colonoscopy, or 2) normal breakfast, low residue lunch, and then clear liquids through the remainder of the day prior to the exam. There were no significant differences with either diet with 93-95% reported as good to excellent preparation. However, the subjects consuming the low residue lunch reported less hunger and more energy, therefore permitting them to perform their usual daily activities.

Delegge randomized 506 colonoscopy patients to receive either: 1) clear liquid diet and sodium phosphate prep, or 2) NeutraPrep, a low residue meal kit with magnesium citrate colon preparation. Both preparations resulted in greater than 80% good or excellent colon cleansing with a slight increase, 85% versus 73%, in subjects that would repeat this preparation for a future exam.

Rapier, et al, studied 114 patients to receive either: 1) clear liquid diet and a laxative kit with mag citrate oral bisacodyl tablets and a bisacodyl suppository; 2) the low residue meal kit with the above laxative kit; or 3) the low residue meal kit with PEG solution. 81% of group 1, 89% of group 2, and 92% of group 3 had a good or excellent colon cleansing rating by the endoscopist. Despite showing the use of low residue diet as safe and effective, however, there were no significant differences in how the patients rated the tolerability of the regimen in this study.

Aoun, et al, took this one step further and evaluated the effectiveness of an unrestricted diet on the day before colonoscopy. 141 patients were randomized to: 1) 4 liter PEG and clear liquid diet the day before the procedure, or 2) 2 liter PEG and regular diet (up until 6:30 P.M. the day before the procedure) and 2 liter PEG on the morning of the procedure. The clear liquid group had 56.2% good to excellent preparation compared to 76.5% in the regular diet group. The regular diet group also demonstrated a non-significant trend towards increased compliance (90% versus 78%).

Another significant and well described problem is the movement of ileal chyme into the cecum and right colon if the last laxative is ingested greater than 6 hours before the procedure time. This chyme effluent is very adherent to the colonic mucosa and difficult to irrigate or suction, making visualization of the right colon suboptimal. Split dosing of the laxatives (sodium phosphate and PEG) by giving the second dose of laxative approximately 4 hours before the procedure, has been shown to provide clearing of this ileal effluent and improved right colon viewing with colonoscopy. Unfortunately, patients scheduled for morning procedures would be required to awaken at 3:00 A.M. and later to ingest another dose of unpleasant laxative. They would then redevelop diarrhea with its associated symptoms throughout the morning hours, with further loss of sleep, GI distress and the practical problem of trying to travel to the endoscopy suite with lingering GI symptoms and possible incontinence. It is the practice of some institutions and endoscopists to routinely give split dose prep instructions of either sodium phosphate or PEG, to optimize bowel preparation. A large number of patients, however, are still given same day prep directions to prepare their colon on the day before their procedure to minimize the already unpleasant and cumbersome task of tolerating colon preparation.

Patents and Patent Publications

Review of the prior art reveals a number of patents concerned with bowel health and bowel preparations. Most of these bowel preparations have been discussed above. Two such patents, U.S. Pat. No. 6,866,873, and U.S. Pat. No. 7,282,223, describe a nutritional dietary system, formulation, kit and method for use in preparing an individual for a predetermined activity.

In reviewing the past studies, and from clinical experience with approximately 15,000 to 17,000 colonoscopy patients, the importance, yet difficulties of colonoscopy preparation are well documented. Thus, a typical statement heard from patients is, “the prep was way worse than the procedure”. Therefore, a method and kit that facilitates good colon prep with minimal patient disruption is desirable.

### SUMMARY OF THE INVENTION

A colon prep method and kit for facilitating the method for evacuating a colon for a medical procedure, such as a colonoscopy, wherein the method minimizes potential patient discomfort. The method includes the steps of ingesting three selected meals and a plurality of selected snacks on a day prior to the medical procedure. The meals include solid food items that are provided with a stimulant laxative or that have a stimulant laxative incorporated therein. The stimulant laxative is preferably senna. Additionally, the meals include a liquid having a PEG laxative incorporated therein. The PEG laxative is preferably PEG-3350. In the kit of the invention, a powdered drink mix is preferably provided. The stimulant laxative and the PEG laxative work in synergy.

A further optional snack may be provided on the day of the medical procedure. The same day snack includes a solid food item provided with a stimulant laxative or that have a stimulant laxative incorporated therein. The same day snack additionally includes a liquid having a PEG laxative incorporated therein. Preferably, the same day snack is ingested approximately four hours before the medical procedure.

In a preferred method, the three selected meals include a meal package containing approximately 70 mg of senna and a drink mix containing approximately 68 gm of PEG-3350. The senna in the meal package may be provided with a solid food item or may be incorporated into a solid food item such as a muffin, oatmeal, pasta with sauce, beef or chicken based soup with crackers.

The PEG-3350 in the drink mix is preferably incorporated into a drink item such as a cran-apple drink mix, lemonade drink mix. The snack package preferably contains approximately 26-36 mg of senna and the drink mix of the snack package preferably contains approximately 68 gm of PEG-3350. The senna in the snack package is preferably incorporated into a food item such as chocolate pudding, a chocolate bar, or a chocolate Rice Krispies treat.

### BRIEF DESCRIPTION OF THE DRAWINGS

FIG. 1 shows a schematic diagram of the kit of the invention.

### DETAILED DESCRIPTION OF THE PREFERRED EMBODIMENTS

In light of the shortcomings associated with known preps and diet regimens, the kit and method of the invention provides a simple solution. Safe laxative ingredients are provided with food and drinks or may be placed into food and drinks in a stepped residue diet kit that offers simplicity, good taste, affordability, and effective bowel cleansing. In one embodiment, senna, which does have a bitter taste, is prepared in solid foods to produce stimulation of gut motility. Powdered PEG-3350 is dispersed in fluids to produce an osmotic lavage of the bowel contents. These two agents work in concert with each other to produce bowel cleansing. While the idea of mixing medicine with a teaspoon of sugar is an old concept, the kit of the invention uses a specific type of food and drink combination with specific carbohydrate, protein and fat mixtures, fiber content, preparation temperatures, color, and textures with appropriate laxative doses.

Senna has a bitter taste and brownish color and, therefore, may be prepared with foods of specific corresponding color, acidity, taste, and texture to disguise both of these unpleasant characteristics. Senna also undergoes degradation above certain preparation temperatures, requiring lower temp/quicker preparation type foods.

The laxative dosage must also be specific and graded to provide a minimal needed amount to produce adequate laxative effect versus a supra-therapeutic amount that tends to produce abdominal cramps and pain. The stimulation of senna, either administered with or incorporated into solid foods will work in synergy with the osmotic laxative effect of PEG-3350 in the fluids ingested allowing a smaller effective dose of both agents.

The preparation of the invention involves frequent small doses of senna, either administered with food products or hidden in the food products, i.e., doses of 0-64 mg in the main meals, alternating with 0-34 mg of senna administered in or with snacks to be every 2-3 hours, again in the form of breakfast, lunch, dinner and snacks with a total dose of 108 mg. In greater detail, 0-125 mg of senna, as part of the inventive regimen, is believed to be effective for meal dosing with 12.5-64 mg senna being preferred and 37.5 mg senna being more preferred.

The timing of meals, snacks and drinks is also of considerable importance due to the requirement of contact with colonic bacteria to degrade senna to its active form. This delay from oral ingestion to effect will be of longer duration after the first meal (3-5 hours) than the last meal (0.5 to 1 hour) because of increased bowel motility as subsequent laxatives are ingested. The bulk of stooling and preparation of the colon should be completed by 9 to 10 o'clock of the night before procedure.

PEG-3350 powder (MiraLax®, GlycoLax®) is a powder that, when mixed with liquid, becomes tasteless, odorless, colorless and clear. It does alter the composition of solid foods, however, and therefore is restricted in the kit of the invention to liquid drink mixes. PEG-3350 produces an osmotic laxative effect by retention and excretion of fluid into the bowel with associated bowel distention, contractions and subsequent defecation. PEG-3350 can be associated with abdominal fullness and bloating due to its physiologic action. As noted before, however, it will work synergistically with senna, allowing our use of lower doses. In the prep of the invention, 17-34 grams of PEG-3350 are mixed per drink for a total of 136 to 153 grams. In greater detail, 0-102 g of PEG-3350, as part of the inventive regimen, is believed to be effective for meal dosing with 17-68 g being preferred and 34 g being most preferred.

Having the option of a split dose laxative within the kit of the invention also provides a unique element over other bowel preparations, namely the ability to individualize the preparation to the patient's typical bowel habits. All previous bowel preparations follow a similar yet flawed assumption, one size fits all. There are typically no provisions for the variety of bowel habits that are seen in a patient population. Therefore, for example, four liters of Golytely may be too little for some patients and too much for the next patient, and so on for all of the currently available bowel preparations. In addition to the physical discomfort of too much preparation with its associated cramps, nausea, bloating, diarrhea, and perirectal pain, patients experience emotional distress over presenting for an embarrassing exam with a not fully clean colon. The greater risk actually lies in the inability to adequately visualize a poorly prepped colon, therefore increasing the patient's risk that polyps and other lesions may be missed. Lastly, studies have shown that poor preps lead to shorter recommended screening intervals, more procedures, and greater healthcare costs.

The inventive kit includes a time appropriate snack with a drink laced with senna and PEG-3350, respectively, or taken with senna and PEG-3350, to be given 4 hours before the procedure depending on the endoscopist's preference, patient's bowel history (e.g., history of chronic constipation) and whether the patient senses incomplete bowel evacuation with the previous day's diet and prep (e.g., abdominal rectal fullness or cloudy stool effluent on the morning of the exam). Patients scheduled for afternoon cases could also use the snack and drink 4 hours before the afternoon times with no undue discomfort of awakening in early morning hours.

Both senna and MiraLax® (PEG-3350), appear to be very safe with numerous reports showing no significant changes in electrolytes, EKG's or clinical symptoms. This is opposed to frequent electrolyte alterations and infrequent, but extremely dangerous, hyperphosphatemia with renal damage associated with sodium phosphate colon preparations. This has lead to Fleets phosphosoda being removed from the OTC shelves in 2008 and now being available by prescription only. Fleets phosphosoda and the other current prescription phosphosoda tablets now carry a blackbox warning concerning the risk of kidney damage. This will likely significantly reduce their use as bowel preparation agents further limiting patient's choices of safe bowel preparation agents. The other factors that are more difficult to estimate but documented to be barriers to colonoscopy including the cost of some laxatives (Half Lightly can cost in the range of $60-$80), the difficulty of understanding a “clear liquid diet” instruction sheet, difficulty in preparing a clear liquid diet, and possibly most important, the willpower to “just have liquids” or “fast” for 24-36 hours before the procedure.

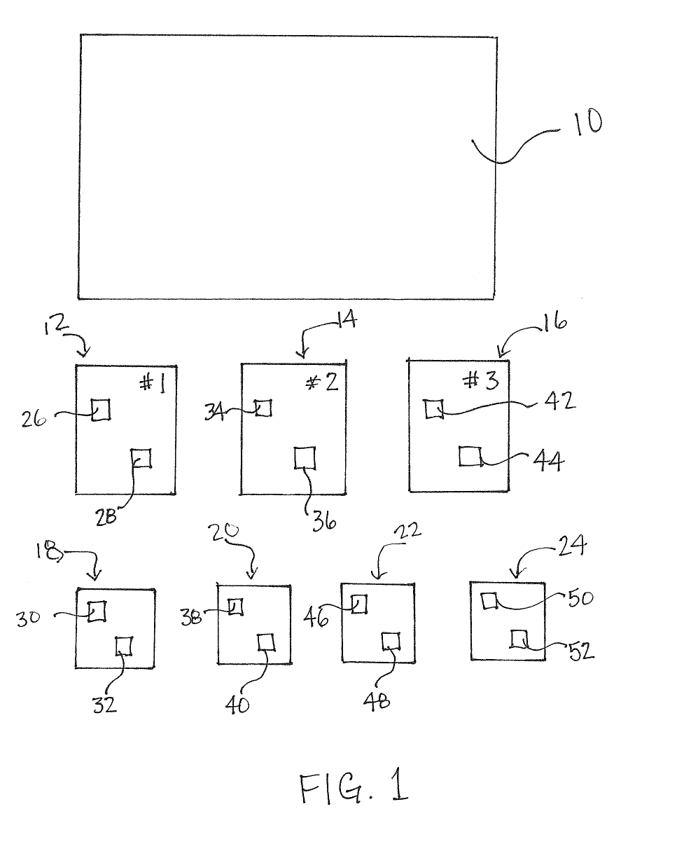

Referring now to FIG. 1, the inventive diet preparation kit costs approximately $25-$35, requires no understanding of clear liquid diets, is packaged to simplify preparation of meals and drinks so that meals need only to be mixed with water or heated less than 90 seconds in a microwave and don't require “fasting or not eating”. In a preferred embodiment, the kit is packaged in a single box 10 with a carrying handle. Within box 10 is a package 12 labeled #1 for breakfast, a package 14 labeled #2 for lunch, and a package 16 labeled #3 for dinner. Three snacks 18, 20, 22 are provided that are associated with breakfast, lunch and dinner. The snacks 18, 20, 22 are labeled as morning snack, afternoon snack and evening snack. Additionally, an optional snack package 24, containing a food and drink mix are provided for ingesting 4 hours before the procedure.

Package 12, containing breakfast kit #1 preferably includes typical breakfast foods 26 and drink mixes 28. Package 12 may include, but is not limited to, a blueberry muffin with 0-36 mg of senna, an oatmeal package with 0-36 mg of senna and a cran-apple drink mix for mixing with 12 ounces of water. The drink mix 28 preferably has 0-68 gram of PEG-3350. Package 18, containing a morning snack is preferably a solid food item 30, such as typical snack food. Package 18 may include, but is not limited to, chocolate pudding with 0-36 mg of senna. Package 18 preferably contains a drink mix 32 containing PEG-3350

Package 14, containing the lunch kit, preferably includes typical lunch foods. Package 14 preferably includes, but is not limited to, food item 34, such as a pasta with sauce having 0-36 mg of senna, and drink mix 36, such as a lemonade drink mix with 0-68 grams of PEG-3350. Package 20, containing the afternoon snack preferably contains a solid food item 38, such as a typical snack food. Package 20 preferably includes, but is not limited to, a chocolate bar with 0-36 mg of senna.

Package 16, i.e., the dinner kit, preferably includes solid food items 42, such as typical dinner foods. Package 16 preferably includes, but is not limited to, a beef or chicken based soup with crackers, 0-36 mg of senna in the soup, and a drink mix 44 with 0-68 grams of PEG-3350. Every snack package 22 preferably contains solid food item 46 and a drink mix item 48.

Package 24, containing an optional snack used for split dose prepping, preferably includes a solid food item 50, such as a typical snack food. The snack is preferably, but is not limited to, a chocolate Rice Krispies® treat with 0-36 mg senna and a drink mix 52 with 0-68 grams of PEG-3350. All meals and snacks are preferably packaged in one box with a carrying handle and prep directions on the inside and outside of the box.

The present invention is a significant advance over the prior art, by permitting the patient to be on a much more liberal diet on the day before a procedure than is typically permitted. This should markedly lessen patients discomfort associated with clear liquid and low residue diets. The method and kit of the invention requires essentially no willpower, the lack of which interferes with many patients' colon preparations.

Secondly, and probably of much more importance, the method and kit of the invention address the worst part of colon preparation, the prep itself. By using foods and drinks with specific tastes, colors, textures, acidity, quantities and preparation qualities, a stimulant and low volume PEG agent can be taken separately or incorporated directly into food and drink in a manner which preserves food and drink palatability. Furthermore, by using a specific quantity and combination of stimulant and PEG agent at specific times, less quantity of each laxative is needed, markedly lessening the discomfort typically associated with a laxative when used as single agents to prepare the colon.

Clinical Observations

Support for the concept of the invention includes experience resulting from some 19 years of didactic training, literature review and clinical experience of the inventor. Historically, patients are placed on a clear liquid diet for 24-48 hours prior to their colonoscopy while they simultaneously ingest their colon prep. In the course of performing over 20,000 colonoscopies, many patients have presented with GI symptoms necessitating colonoscopy on the following day. Patients have typically been on a regular diet for breakfast and lunch prior to beginning their colon prep. The prep would then commence in the afternoon or evening prior to their procedure the next day. Despite this non-adherence to a clear liquid diet for the entire day of colon preparation, they present for colonoscopy usually with very satisfactory colon cleansing. It is clear that a more liberal diet on the prep day can still allow an adequate preparation of the colon.

In addition to multiple anecdotal cases, observational studies were performed with portions or an entire meal plan in accordance with the invention. A stepped phase approach to trials of various meals beginning with a single meal/laxative along with a drink mix/PEG3350 laxative were conducted, progressing to a complete prep process that included: breakfast, lunch, dinner, snacks and drinks with laxatives. Participants were asked which of the following most accurately described their most predominate bowel pattern prior to beginning the prep: constipated (less than 1 bowel movement every 3 days), normal (from 1 bowel movement every 3 days to 3 bowel movements per day), and frequent (more than 3 bowel movements per day).

Participants were given instructions on how to prepare the meals and drink mixes. Each meal and drink were consumed together at one sitting. Comments were then recorded as to the ease of preparation using a rating of 1 to 4 selected from the following: easy to prepare (score 1), moderate difficulty to prepare (score 2), and very difficult to prepare (score 3). Palatability of the meal and drink were each rated from 1 to 3: good taste (score 1), fair taste (score 2), and bad taste (score 3).

A stool diary was kept that described the number and consistency of bowel movements (hard, formed, soft, loose or watery) following ingestion of the prep. The presence of side effects (abdominal pain, gas, bloating, nausea and vomiting) was rated on a scale from 1 to 4: absent (score 1), mild (score 2), moderate (score 3) and severe (score 4).

Experimental Results:

Participant #1—received breakfast meal kit containing dry oats flavored with butter buds, cinnamon, and Splenda®, mixed with 25 mg of senna, in addition to a dry cran-apple powdered drink mixed with 54 gm of Miralax®. The participant was given instructions to mix the dry oats with one cup of water and heat with the microwave on high for 90 seconds. The powdered drink was to be mixed with 16 oz of water for one minute.

Participant #1 rated the food and drink mix both 1 for ease of preparation, both 1 for taste, and 1 for side effects. Stools began within 1 hour of finishing the meal and drink. Initially the stool was formed and then progressed to loose but not watery for a total of 5 bowel movements. Participant #1 reported normal pre-prep bowel habits.

Participant #2—received a lunch meal kit containing an Italian style pasta bake with 25 mg of senna and a lemonade powdered drink with 34 grams of PEG 3350 mixed with 16 oz of water. The pasta bake was heated for 90 seconds in a conventional microwave.

Participant #2 rated the food and drink mix both 1 for ease of preparation, both 1 for taste, and 2 for side effects. Side effects were described as some mild gas 6 hours after the prep. No bloating or cramping were noted. Stools began 2 hours after finishing the meal and drink. Two bowel movements were produced and were both formed. Participant #2 reported normal pre-prep bowel habits.

Participant #3—received a lunch meal kit containing the same ingredients as participant #2.

Participant #3 rated the food and drink mix both 1 for ease of preparation, both 1 for taste, and 2 for side effects. Side effects were described as gas 5-6 hours after the prep. Two bowel movements were produced and were both formed. Participant reported constipated pre-prep bowel habits.

Participant #4—received a breakfast meal kit and a lunch meal kit. The breakfast kit included the dry oats with seasonings and 25 mg of senna. A powdered orange drink was mixed with 51 gm of PEG 3350 with 16 oz of water. The lunch kit included spaghetti with meat sauce and 37.5 mg of senna along with a lemonade drink mixed with 51 gm of PEG 3350 and 16 oz of water. Preparation instructions were given.

Participant #4 rated the food and drink mixes for breakfast and lunch as both 1 for ease of preparation, both 1 for taste, and 2 for side effects. Side effects were described as some mild upper abdominal pain and gas beginning 5.5 hours after the lunch meal. Bowel movements began 8 hours after the lunch meal and initially were hard and progressed to loose by the next morning (22 hours later), approximately 6-8 bowel movements in total. Participant reported constipated pre-prep bowel habits.

Participant #5—received a dinner kit containing spaghetti with meat sauce and 37.5 mg of senna along with a powdered cranberry drink mixed with 34 gm of PEG 3350 and 16 oz of water. Preparation instructions were given.

Participant #5 rated the food and drink mix both as 1 for ease of preparation, both 1 for taste, and 1 for side effects. Bowel movements began 2 hours after the dinner was ingested and were hard initially and progressed to formed, 2 bowel movements total. Participant reported constipated pre-prep bowel habits.

Participant #6—received a breakfast kit containing flavored oats and 25 mg of senna with toast, along with a powdered orange drink mixed with 34 gm of PEG 3350 and 16 oz of water. The participant received a lunch kit containing a soup consisting of meat, potato and bean with crackers along with a powdered raspberry drink mixed with 34 gm of PEG 3350 and 16 oz of water. The participant received a dinner kit containing spaghetti and 37.5 mg of senna along with a powdered lemonade drink mixed with 34 gm of PEG 3350 and 16 oz of water. Participant received a mid-morning snack consisting of chocolate pudding and a mid-afternoon snack consisting of a chocolate Rice Krispies® bar, each with 25 mg of senna. The participant was allowed additional fluids as desired and instructions were given for the meal kits and snacks.

Participant #6 rated all of the food and drink mixes as 1 for ease of preparation, 1 for taste, and 1 for side effects. Both snacks were rated as 2 for taste. Bowel movements began 3.25 hours after the first meal kit with formed stool and progressed to loose stool. A total of 14 stools were reported during and immediately after the prep period. Participant reported normal pre-prep bowel habits.

Participant #7—received the same meal kits and snacks as participant #6. The participant was allowed additional fluids as desired and instructions were given for the meal kits and snacks.

Participant #7 rated all of the food and drink mixes as 1 for ease of preparation. Participant rated breakfast, mid-morning snack and lunch meals a 2; mid-afternoon snack and dinner meal a 1 for taste. Side effects were rated at 1. Bowel movements began 8.5 hours after the first meal kit with loose stool and progressed to watery. Participant reported multiple bowel movements. Participant reported constipated pre-prep bowel habits.

Participant #7 underwent colonoscopy on the following morning. The procedure was carried out using standard protocol with IV conscious sedation (midazolam and meperidine) by an unblinded board certified endoscopist. Total procedure time and ileocecal intubation were recorded. Global colon cleansing was rated using an Aron-chick scoring scale (1-excellent, 2-good, 3-fair, and 4-inadequate). Colon cleansing was rated as a 1 by the unblinded endoscopist.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A colon prep method for evacuating a colon for a medical procedure wherein the method minimizes potential patient discomfort, the method comprising the steps of:
ingesting three selected meals and a plurality of selected snacks on a day prior to the medical procedure;
wherein said meals comprise a solid food item having a stimulant laxative provided therewith;
wherein said meals comprise a liquid having a PEG laxative provided therewith.

2. The method of claim 1 wherein:
said stimulant laxative is incorporated in said solid food item.

3. The method of claim 1 wherein:
said PEG laxative is incorporated in said liquid.

4. The method of claim 1 further comprising the steps of:
ingesting a snack on the day of the procedure;
wherein said snack comprises a solid food item having a stimulant laxative provided therewith;
wherein said snack comprises a liquid having a PEG laxative provided therewith.

5. The method of claim 4 wherein:
said stimulant laxative is incorporated in said solid food item.

6. The method of claim 4 wherein:
said PEG laxative is incorporated in said liquid.

7. The method of claim 1 wherein said solid food item, said liquid, said stimulant laxative and said PEG laxative are ingested approximately four hours before the medical procedure.

8. The method of claim 1 wherein said stimulant laxative of said meals is comprised of senna.

9. The method of claim 1 wherein said PEG laxative of said meals is PEG 3350.

10. The method of claim 4 wherein said stimulant laxative of said snack is comprised of senna.

11. The method of claim 4 wherein said PEG laxative is PEG-3350.

12. The method according to claim 1 wherein:
said stimulant laxative and said PEG laxative work in synergy.

13. The method according to claim 1 wherein said three selected meals comprise:
a meal package comprising approximately 70 mg of senna;
a drink mix comprising approximately 68 gm of PEG-3350.

14. The method according to claim 1 wherein said three selected meals comprise:
a meal package comprising approximately 125 mg of senna;
a drink mix comprising approximately 102 gm of PEG-3350.

15. The method according to claim 1 wherein said three selected meals comprise:
a meal package comprising approximately 12.5-64 mg of senna;
a drink mix comprising approximately 17-68 gm of PEG-3350.

16. The method according to claim 1 wherein said three selected meals comprise:
a meal package comprising approximately 0-64 mg of senna;
a drink mix comprising approximately 17-34 gm of PEG-3350.

17. The method according to claim 1 wherein said three selected meals comprise:
a meal package comprising senna;
wherein the senna in the meal package is incorporated into a solid food item selected from the group consisting of: muffin, oatmeal, pasta with sauce, beef or chicken based soup with crackers.

18. The method according to claim 1 wherein said three selected meals comprise:
a drink mix comprising PEG-3350;
wherein the PEG-3350 in the drink mix is incorporated into a drink item selected from the group consisting of: cran-apple drink mix, lemonade drink mix.

19. The method according to claim 1 wherein said plurality of selected snacks comprise:
a snack package containing approximately 26-36 mg of senna;
a drink mix comprising approximately 68 gm of PEG-3350.

20. The method according to claim 1 wherein said plurality of selected snacks comprise a snack package wherein the senna in the snack package is incorporated into a food item selected from the group consisting of: chocolate pudding, chocolate bar, chocolate Rice Krispies treat.

21. A colon prep kit for facilitating evacuating a colon for a medical procedure, said kit comprising:
three pre-packaged meals;
wherein said pre-packaged meals are comprised of a solid food item and a stimulant laxative provided therewith;
wherein said pre-packaged meals are comprised of a drink mix and a PEG laxative provided therewith.

22. The kit according to claim 21 wherein:
said stimulant laxative is incorporated in said solid food item.

23. The kit according to claim 21 wherein:
said PEG laxative is incorporated in said drink mix.

24. The kit of claim 21 wherein said stimulant laxative is comprised of senna.

25. The kit of claim 21 wherein said PEG laxative is PEG-3350.

26. The kit of claim 21 further comprising:
a plurality of pre-packaged snacks;
wherein said pre-packaged snacks are comprised of a solid food item and a stimulant laxative provided therewith; and
wherein said pre-packaged snacks are comprised of a drink mix and a PEG laxative provided therewith.

27. The kit according to claim 26 wherein:
said stimulant laxative is incorporated in said solid food item.

28. The kit according to claim 26 wherein:
said PEG laxative is incorporated in said drink mix.

29. The kit of claim 26 wherein said stimulant laxative is comprised of senna.

30. The kit of claim 26 wherein said PEG laxative is PEG-3350.

31. The kit according to claim 26 wherein:
said stimulant laxative and said PEG laxative work in synergy.

32. The kit according to claim 26 wherein said three pre-packaged meals are comprised of:
said solid food item having approximately 0-64 mg of senna provided therewith; and
said drink mix provided with approximately 17-34 gm of PEG-3350.

33. The kit according to claim 26 wherein said three pre-packaged meals are comprised of:
said solid food having approximately 0-125 mg of senna provided therewith; and
said drink mix provided with approximately 0-102 gm of PEG-3350.

34. The kit according to claim 26 wherein said three pre-packaged meals are comprised of:
said solid food having approximately 12.5-64 mg of senna provided therewith; and
said drink mix provided with approximately 17-68 gm of PEG-3350.

35. The kit according to claim 26 wherein said three pre-packaged meals are comprised of:
said solid food having approximately 37.5 mg of senna provided therewith; and
said drink mix provided with approximately 34 gm of PEG-3350.

36. The kit according to claim 21 wherein said stimulant laxative in the pre-packaged meal is incorporated into a solid food item selected from the group consisting of: muffin, oatmeal, pasta with sauce, beef or chicken based soup with crackers.

37. The kit according to claim 21 wherein the PEG-3350 in the drink mix is incorporated in a drink mix selected from a group comprising: cran-apple drink mix, lemonade drink mix.

38. The kit according to claim 26 wherein said pre-packaged snacks are comprised of:
solid food items incorporating approximately 26-36 mg of senna;
a drink mix comprising approximately 68 gm of PEG-3350.

39. The kit according to claim 38 wherein said food item in the pre-packaged snacks incorporates a solid food item selected from a group consisting of: chocolate pudding, chocolate bar, chocolate Rice Krispies treat.
